(19) United States
(12) Patent Application Publication
(10) Pub. No.: US 2021/0330262 A1
(43) Pub. Date: Oct. 28, 2021

(54) BIOINFORMATICS SENSOR PATCH

(71) Applicant: Seknova Biotechnology Co., Ltd., Taipei City (TW)

(72) Inventors: CHING CHUN HUANG, Hsinchu (TW); SHIH HSUAN KU, Hsinchu (TW); YI JEN CHEN, Hsinchu (TW); PING CHUN WANG, Hsinchu (TW)

(21) Appl. No.: 17/236,547

(22) Filed: Apr. 21, 2021

Related U.S. Application Data
(60) Provisional application No. 63/014,335, filed on Apr. 23, 2020.

(51) Int. Cl. A61B 5/00; A61B 5/01; A61B 5/145

(52) U.S. Cl. CPC A61B 5/6833; A61B 5/6844; A61B 5/7405; A61B 5/742; A61B 2560/0252; A61B 5/01; A61B 5/14503; A61B 5/14532; A61B 5/14546; A61B 5/7455

(57) ABSTRACT

A bioinformatics sensor patch includes a plurality of sensing electrodes, a distance sensing element and an operation unit. The sensing electrodes senses bioinformatics of an organism. The distance sensing element senses a contact degree between the bioinformatics sensor patch and the organism and generates a corresponding contact signal. The operation unit is electrically connected to the sensing electrodes and the distance sensing element to receive the bioinformatics and the contact signal, wherein the operation unit compensates the bioinformatics or selectively outputs a control signal according to the contact signal. The above-mentioned bioinformatics sensor patch can improve a sensing accuracy of the bioinformatics.

### BACKGROUND OF THE INVENTION

### 1. Field of the Invention

The present invention relates to a sensor, particularly to a bioinformatics sensor patch.

### 2. Description of the Prior Art

In the conventional method of acquiring bioinformatics (such as blood glucose), the medical personnel uses a syringe to suck blood of the testee or the testee himself punctures his skin to obtain a small amount of blood for chemical analysis. The conventional method can only acquire the bioinformatics occurring at a single time point. If the tests are undertaken very frequently in a short interval of time, the operator should feel inconvenient, and the testee would suffer more pain. Thus, the testees are likely to refuse too frequent tests. Then, the number of tests will be reduced.

So far, bioinformatics sensor patches have been developed to overcome the abovementioned problem. The bioinformatics sensor patch is attached to the skin of an organism and uses low-invasiveness punctures to reduce pain of the testee and acquire the tissue fluid of the testee. Thus, the bioinformatics sensor patch can obtain the bioinformatics of the testee continuously in a longer term. However, inappropriate attachment operation by the operator or variation of the external environment may result in measurement errors of the bioinformatics sensor patch.

Hence, improving the measurement accuracy of bioinformatics sensor patches has become the target the concerned fields are eager to achieve.

### SUMMARY OF THE INVENTION

The present invention provides a bioinformatics sensor patch, which comprises a distance sensing element for sensing a contact degree between the bioinformatics sensor patch and an organism, whereby to compensate for the sensed bioinformatics or remind the user to eliminate an abnormality. Therefore, the present invention can improve the accuracy of sensing bioinformatics.

In one embodiment, the bioinformatics sensor patch of the present invention comprises a plurality of sensing electrodes, a distance sensing element and an operation unit. The plurality of sensing electrodes contacts an organism to sense bioinformatics of the organism. The distance sensing element is used to sense a contact degree between the bioinformatics sensor patch and the organism and generate a corresponding contact signal. The operation unit is electrically connected with the plurality of sensing electrodes and the distance sensing element to receive the bioinformatics and the contact signal, wherein the operation unit compensates for the bioinformatics or selectively outputs a control signal according to the contact signal.

The objective, technologies, features and advantages of the present invention will become apparent from the following description in conjunction with the accompanying drawings wherein certain embodiments of the present invention are set forth by way of illustration and example.

### BRIEF DESCRIPTION OF THE DRAWINGS

The foregoing conceptions and their accompanying advantages of this invention will become more readily appreciated after being better understood by referring to the following detailed description, in conjunction with the accompanying drawings, wherein

FIG. 1 is a diagram schematically showing a bioinformatics sensor patch according to a first embodiment of the present invention;

FIG. 2 is a diagram schematically showing a bioinformatics sensor patch according to a second embodiment of the present invention;

FIG. 3 is a diagram schematically showing a bioinformatics sensor patch according to a third embodiment of the present invention;

FIGS. 4a-4c are diagrams schematically showing the structure of the distance sensing element, the first temperature sensor, and the second temperature sensing electrode of the bioinformatics sensor patch of the present invention according to one embodiment of the present invention;

FIG. 5 is a flowchart of a bioinformatics compensation method of the bioinformatics sensor patch according to the first embodiment of the present invention;

FIG. 6 is a flowchart of a bioinformatics compensation method of the bioinformatics sensor patch according to the second embodiment of the present invention;

FIG. 7 is a flowchart of a bioinformatics compensation method of the bioinformatics sensor patch according to the third embodiment of the present invention;

FIG. 8 is a flowchart of another bioinformatics compensation method of the bioinformatics sensor patch according to the third embodiment of the present invention;

FIG. 9 and FIG. 10 show relationships between the capacitance, resistance and voltage and the distance between the bioinformatics sensor patch of the present invention and a contact surface;

FIG. 11 shows a relationship between the contact degree of the bioinformatics sensor patch of the present invention and a first compensation parameter;

FIG. 12 shows a relationship between the ambient temperature of the bioinformatics sensor patch of the present invention and a second compensation parameter;

FIG. 13 shows a relationship between the body temperature detected by the bioinformatics sensor patch and a third compensation parameter.

### DESCRIPTION OF THE PREFERRED EMBODIMENTS

Various embodiments of the present invention will be described in detail below and illustrated in conjunction with the accompanying drawings. In addition to these detailed descriptions, the present invention can be widely implemented in other embodiments, and apparent alternations, modifications and equivalent changes of any mentioned embodiments are all included within the scope of the present invention and based on the scope of the Claims. In the descriptions of the specification, in order to make readers have a more complete understanding about the present invention, many specific details are provided; however, the present invention may be implemented without parts of or all the specific details. In addition, the well-known steps or elements are not described in detail, in order to avoid unnecessary limitations to the present invention. Same or similar elements in Figures will be indicated by same or similar reference numbers. It is noted that the Figures are schematic and may not represent the actual size or number of the elements. For clearness of the Figures, some details may not be fully depicted.

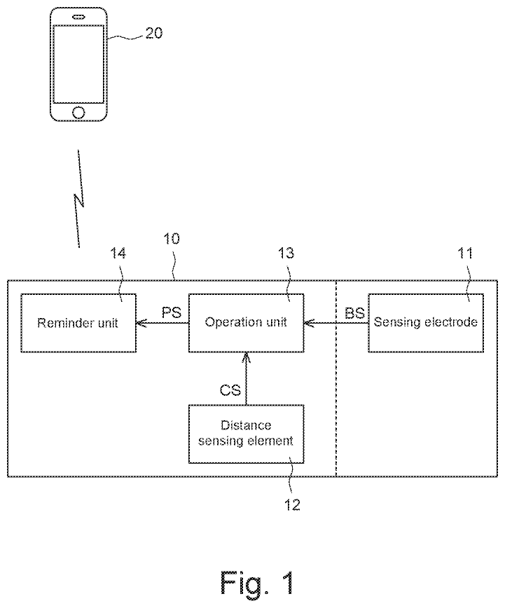

Refer to FIG. 1. In one embodiment, the bioinformatics sensor patch 10 of the present invention comprises a plurality of sensing electrodes 11, a distance sensing element 12 and an operation unit 13. The plurality of sensing electrodes 11 contacts an organism to sense bioinformatics BS of the organism. The plurality of sensing electrodes 11 may contact a tissue layer or a surface of skin to function as working electrodes or reference electrodes. For example, the plurality of sensing electrodes 11 includes at least one reference electrode and at least one working electrode. The plurality of working electrodes may be respectively coated with different sensing layers to sense different bioinformatics BS. In one embodiment, the plurality of electrodes 11 includes at least one working electrode, at least one reference electrode and at least one counter electrode.

The sensing layer coated on the sensing electrode 11 may convert bioinformatics BS into an electronic signal. For example, the bioinformatics may be at least one of concentrations of blood glucose, blood ketone, lactic acid, an antibody, and a drug. The sensing layer may include a complex component or a multilayer structure. For measuring blood glucose, the sensing layer includes glucose oxidase and/or glucose dehydrogenase. In one embodiment, the sensing terminal of the sensing electrode 11 is in form of a sharp needle able to puncture the skin tissue of an organism, whereby the sensing layer coated on the surface of the sensing terminal can react with the tissue fluid or blood of the tissue layer of skin for measuring bioinformatics from the tissue fluid or blood. In one embodiment, the sensing terminal of the sensing electrode 11 is in form of a convex point or a plane, which may contact the surface of skin for measuring bioinformatics of saliva, tear, sweat, or skin surface. In one embodiment, the sensing electrode 11 may be made of an electric-conduction material, such as at least one material of graphene, carbon, gold, silver, stainless steel, nickel, nickel alloys, titanium, and titanium alloys.

The distance sensing element 12 is used to sense a touch degree or a contact degree between the bioinformatics sensor patch 10 and an organism to generate a corresponding contact signal CS. For example, the distance sensing element 12 senses a contact degree between the bioinformatics sensor patch 10 and an organism according to the variation of resistance, capacitance, or voltage (such as piezoelectric elements) and outputs a corresponding contact signal CS. For example, while the user attaches the bioinformatics sensor patch 10 onto his skin, inappropriate operation may cause poor attachment or contact. Besides, frequent movements or collisions may loosen the bioinformatics sensor patch 10. In the abovementioned situations, the distance sensing element 12 may detect abnormality and output a corresponding contact signal CS.

The operation unit 13 is electrically connected with the plurality of sensing electrodes 11 and the distance sensing element 12 to receive the bioinformatics CS sent back by the sensing electrodes 11 and the contact signal CS sent back by the distance sensing element 12. The operation unit 12 may perform a corresponding action according to the contact signal CS sent back by the distance sensing element 12. For example, the operation unit 13 may estimate the depths or uniformity by which the sensing electrodes 11 puncture the skin according to the contact signal CS sent back by the distance sensing element 12, and perform compensation to the bioinformatics BS sent back by the sensing electrodes 11 according to the estimation.

In one embodiment, the bioinformatics sensor patch 10 of the present invention further comprises a reminder unit 14, which is electrically connected with the operation unit 13. While the contact or attachment of the bioinformatics sensor patch 10 to skin is poor, the operation unit 13 may selectively output a control signal PS to control the reminder unit 14 to generate a reminding signal that the user can recognize. For example, the reminder unit 14 may be a display, a light, a buzzer, or a vibration motor, which can directly use text, light, sounds, or vibrations to remind the user of the poor contact between skin and the bioinformatics sensor patch 10, i.e. an abnormality. Then, the user may take an approach to eliminate the abnormality, such as attaching the bioinformatics sensor patch 10 to skin once again. In one embodiment, the reminder unit 14 is a communication interface, whereby the bioinformatics sensor patch 10 can communicate with an external electronic device 20 through the communication interface. Then, the external electronic device 20 reminds the user of the poor contact between skin and the bioinformatics sensor patch 10. For example, the external electronic device 20 is a computer, a mobile Internet-access device, or a dedicated/non-dedicated medical apparatus.

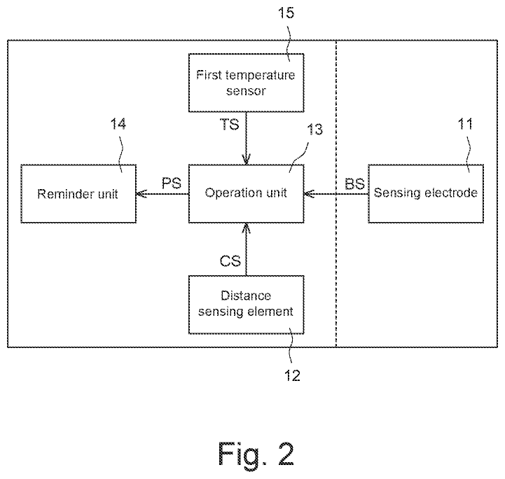

Refer to FIG. 2. In one embodiment, the bioinformatics sensor patch 10 of the present invention further comprises a first temperature sensor 15, which is electrically connected with the operation unit 13. The first temperature sensor 15 is used to sense an ambient temperature TS of the bioinformatics sensor patch 10, whereby the operation unit 13 can take a corresponding action according to the ambient temperature TS sent back by the first temperature sensor 15. For example, while the ambient temperature TS sent back by the first temperature sensor 15 is too high or too low, the operation unit 13 may perform a compensation operation to the bioinformatics BS sent back by the sensing electrodes 11. Alternatively, while the ambient temperature TS sent back by the first temperature sensor 15 is too high or too low, the operation unit 13 may selectively output a control signal PS to control the reminder unit 14 to generate a reminding signal to remind the user to take a measure to improve the problem of ambient temperature, such moving away from the high temperature environment. It is easily understood: the operation unit 13 may simultaneously perform a compensation operation to the bioinformatics BS sent back by the sensing electrodes 11 and selectively output a control signal PS according to the ambient temperature TS sent back by the first temperature sensor 15.

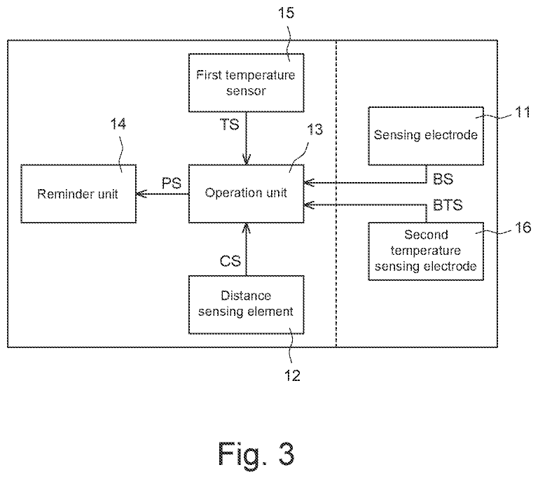

Refer to FIG. 3. In one embodiment, the bioinformatics sensor patch 10 of the present invention further comprises a second temperature sensing electrode 16, which is electrically connected with the operation unit 13. The second temperature sensing electrode 16 may puncture the skin tissue layer of an organism or contact the surface of skin to sense a body temperature BTS of the organism. Similarly, the operation unit 13 may perform a compensation operation to the bioinformatics BS sent back by the sensing electrodes 11 and/or selectively output a control signal PS to remind the user of an abnormality according to the body temperature BTS sent back by the second temperature sensing electrode 16. For example, while the body temperature BTS of the organism is within the range of the working temperature of the sensing electrodes 11, the operation unit 13 performs a compensation operation to the bioinformatics BS sent back by the sensing electrodes 11. While the body temperature BTS of the organism exceeds the range of the working temperature of the sensing electrodes 11, the operation unit 13 selectively outputs a control signal PS to control the reminder unit 14 to remind the user of an abnormality.

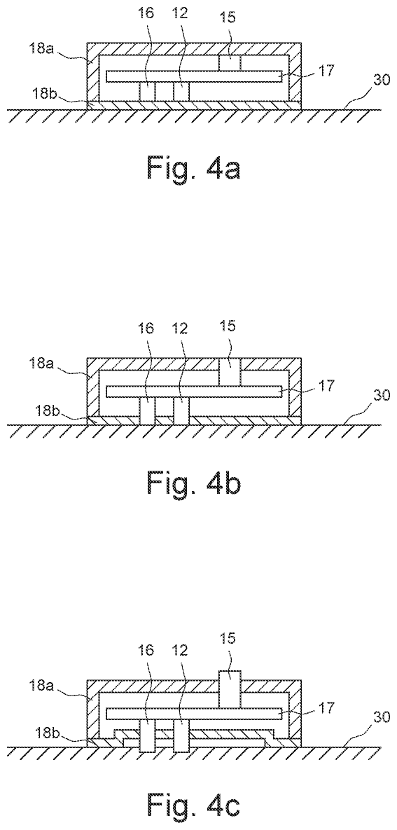

Refer to FIGS. 4a-4c for the structure of the distance sensing element 12, the first temperature sensor 15, and the second temperature sensing electrode 16 of the bioinformatics sensor patch of the present invention. In order to simplify the drawings, the sensing electrodes 11, the operation unit 13 and the reminder unit 14 are omitted in FIGS. 4a-4c. In the embodiments shown in FIGS. 4a-4c, the distance sensing element 12, the first temperature sensor 15 and the second temperature sensing electrode 16 are disposed on a circuit board 17 and electrically connected with the operation unit 13 through the circuit board 17. In the embodiment shown in FIG. 4a, the circuit board 17 is arranged inside a casing formed by an upper cover 18a and a lower cover 18b; the distance sensing element 12, the first temperature sensor 15 and the second temperature sensing electrode 16 on the circuit board 17 are not exposed on the upper cover 18a and the lower cover 18b. For example, the distance sensing element 12 and the second temperature sensing electrode 16 do not contact skin 30 in this embodiment. However, the present invention is not limited by the embodiment. In one embodiment, the distance sensing element 12, the first temperature sensor 15 and the second temperature sensing electrode 16 are exposed on the upper cover 18a or the lower cover 18b, as shown in FIG. 4b. In one embodiment, the distance sensing element 12, the first temperature sensor 15 and the second temperature sensing electrode 16 protrude from the upper cover 18a or the lower cover 18b, as shown in FIG. 4c.

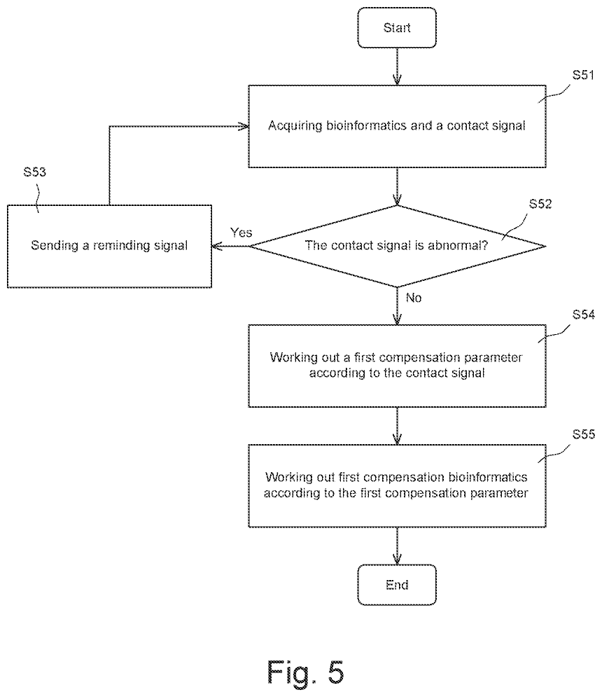

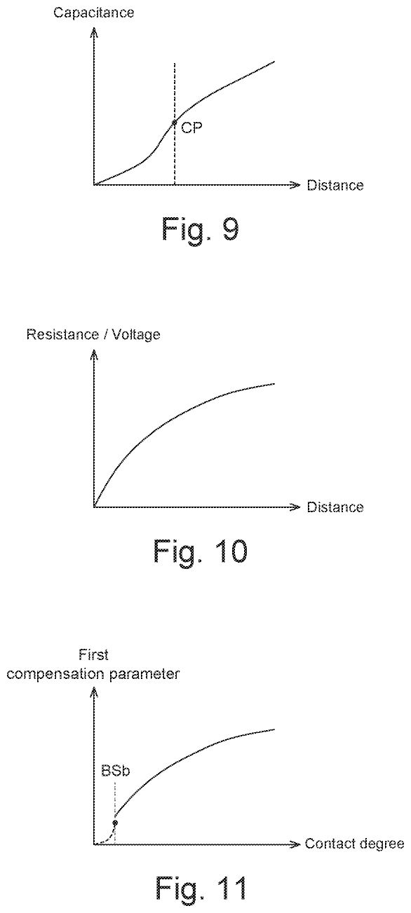

Refer to FIG. 1 and FIG. 5 for a bioinformatics compensation method for a bioinformatics sensor patch according to a first embodiment of the present invention. Firstly, the operation unit 13 acquires the bioinformatics BS detected by the sensing electrodes 11 and the contact signal CS detected by the distance sensing element 12 (S51). Refer to FIG. 9, which shows a relationship between the capacitance and the distance of the bioinformatics sensor patch and the skin, wherein the capacitance varies with the distance. While the capacitance is larger than the capacitance of a contact point CP, the operation unit 13 may determine that the bioinformatics sensor patch of the present invention is normally attached onto the skin. It is easily understood: while the contact pressure is higher, the contact area between the skin and the distance sensing element 12 varies, and the corresponding capacitance also varies. While the distance between the skin and the bioinformatics sensor patch varies, the resistance or voltage also varies, as shown in FIG. 10. The operation unit 13 may determine the contact degree between the skin and the bioinformatics sensor patch of the present invention according to the variation of resistance or voltage.

Next, the operation unit 13 determines whether the contact signal CS is abnormal (S52). If the contact signal CS is abnormal, the operation unit 13 sends a reminding signal through the reminder unit 14 (S53). For example, while the user attaches the bioinformatics sensor patch of the present invention onto his skin, inappropriate operation may cause poor attachment or contact. Besides, frequent movements or collisions may loosen the bioinformatics sensor patch. In the abovementioned situations, the operation unit 13 would find out an abnormality according to the contact signal CS and outputs a reminding signal through the reminder unit 14.

If the contact signal CS is within a preset range, the operation unit 13 works out a first compensation parameter C1 according to the contact signal CS (S54). Refer to FIG. 11, which shows a relationship between the first compensation parameter and the contact degree of the skin and the bioinformatics sensor patch. The operation unit 13 can work out the first compensation parameter C1 according to the gain curve shown in FIG. 11. The first compensation parameter C1 may be expressed by Equation (1):

C1=BSb×Gain (CS) (1)

wherein BSb is the value of the bioinformatics at a specified contact degree, also called the initial condition; Gain (CS) is the gain function of the contact signal CS according to the curve shown in FIG. 11.

Next, the operation unit 13 works out first compensation bioinformatics CBS1 according to the first compensation parameter C1 (S55). The first compensation bioinformatics CBS1 may be expressed by Equation (2):

CBS1=BS/C1 (2)

wherein BS is the bioinformatics detected by the sensing electrodes 11.

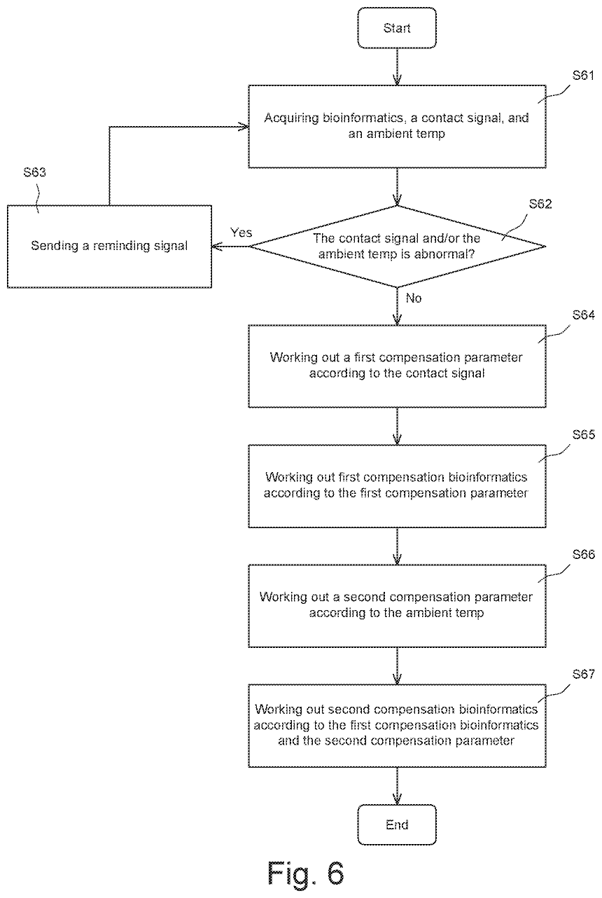

Refer to FIG. 2 and FIG. 6 for a bioinformatics compensation method for a bioinformatics sensor patch according to a second embodiment of the present invention. Firstly, the operation unit 13 acquires the bioinformatics BS detected by the sensing electrodes 11, the contact signal CS detected by the distance sensing element 12 and the ambient temperature TS detected by the first temperature sensor 15 (S61). Next, the operation unit 13 respectively determines whether the contact signal CS is abnormal and whether the ambient temperature TS is abnormal (S62). If the contact signal CS and/or the ambient temperature TS is abnormal, the operation unit 13 sends a reminding signal through the reminder unit 14 (S63).

If the contact signal CS and the ambient temperature TS are within preset ranges, the operation unit 13 works out a first compensation parameter C1 according to the contact signal CS (S64). Next, the operation unit 13 works out first compensation bioinformatics CBS1 according to the first compensation parameter C1 (S65). The calculation methods of the step S64 and the step S65 are the same as those of the step S54 and the step S55 and will not repeat herein.

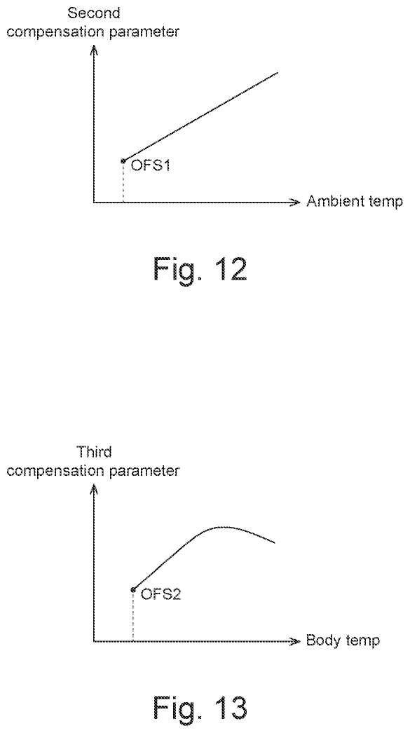

Next, the operation unit 13 works out a second compensation parameter C2 according to the ambient temperature TS (S66). The second compensation parameter C2 is used to compensate for the elements of the bioinformatics sensor patch of the present invention, which are influenced by the ambient temperature. Refer to FIG. 12, which shows the relationship between the second compensation parameter and the ambient temperature of the bioinformatics sensor patch of the present invention. The operation unit 13 can work out the second compensation parameter C2 according to the relationship shown in FIG. 12. The second compensation parameter C2 may be expressed by Equation (3):

C2=M×TS+OFS1 (3)

wherein M is the slope of the straight line shown in FIG. 12; TS is the ambient temperature detected by the first temperature sensor 15; OFS1 is the ambient temperature-induced offset quantity of the measurement system.

Next, the operation unit 13 works out second compensation bioinformatics CBS2 according to the first compensation bioinformatics CBS1 and the second compensation parameter C2 (S67). The second compensation bioinformatics CBS2 may be expressed by Equation (4):

CBS2=Ratio(TS)×CBS1−C2 (4)

wherein Ratio (TS) is to compensate the bioinformatics for the influence of the ambient temperature.

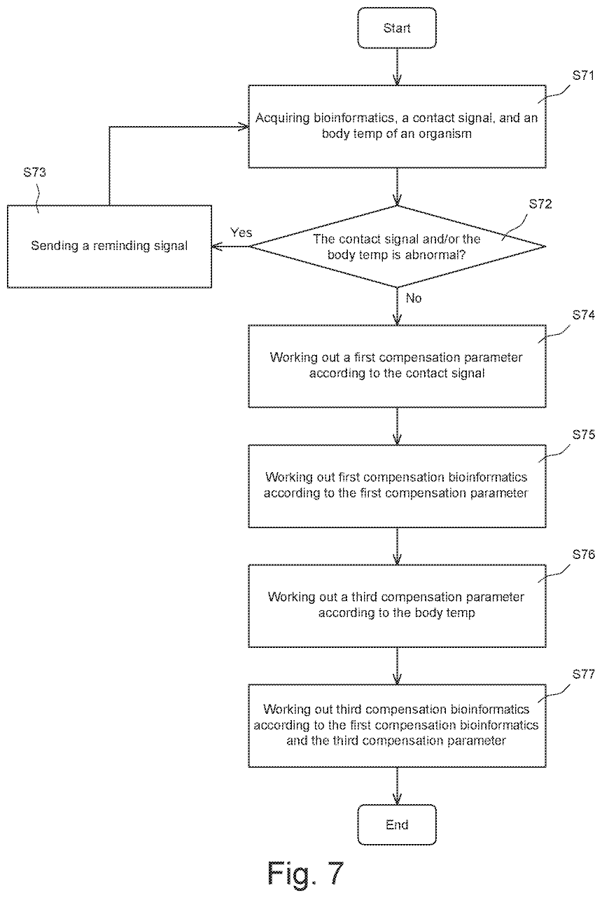

Refer to FIG. 3 and FIG. 7 for a bioinformatics compensation method for a bioinformatics sensor patch according to a third embodiment of the present invention. Firstly, the operation unit 13 acquires the bioinformatics BS detected by the sensing electrodes 11, the contact signal CS detected by the distance sensing element 12 and the body temperature BTS detected by the second temperature sensing electrode (S71). Next, the operation unit 13 respectively determines whether the contact signal CS is abnormal and whether the body temperature BTS is abnormal (S72). If the contact signal CS and/or the body temperature BTS is abnormal, the operation unit 13 sends a reminding signal through the reminder unit 14 (S73).

If the contact signal CS and the body temperature BTS are within preset ranges, the operation unit 13 works out a first compensation parameter C1 according to the contact signal CS (S74). Next, the operation unit 13 works out first compensation bioinformatics CBS1 according to the first compensation parameter C1 (S75). The calculation methods of the step S74 and the step S75 are the same as those of the step S54 and the step S55 and will not repeat herein.

Next, the operation unit 13 works out a third compensation parameter C3 according to the body temperature BTS (S76). Refer to FIG. 13, which shows the relationship between the third compensation parameter and the body temperature. The operation unit 13 can work out the third compensation parameter C3 according to the gain curve shown in FIG. 13. The third compensation parameter C3 may be expressed by Equation (5):

C3=Gain(BTS) (5)

wherein Gain (BTS) is the gain function of the body temperature BTS according to the curve shown in FIG. 13.

Next, the operation unit 13 works out third compensation bioinformatics CBS3 according to the first compensation bioinformatics CBS1 and the third compensation parameter C3 (S77). The third compensation bioinformatics CBS3 may be expressed by Equation (6):

CBS3=CBS1×C3−OFS2 (6)

wherein OFS2 is the body temperature-induced offset quantity of the measurement system.

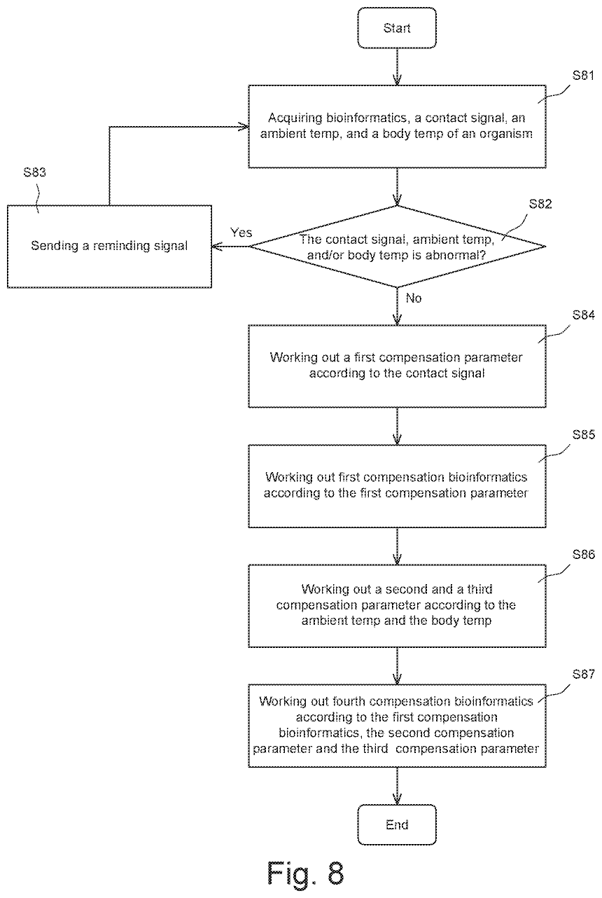

Refer to FIG. 3 and FIG. 8 for another bioinformatics compensation method for a bioinformatics sensor patch according to the third embodiment of the present invention. Firstly, the operation unit 13 acquires the bioinformatics BS detected by the sensing electrodes 11, the contact signal CS detected by the distance sensing element 12, the ambient temperature TS detected by the first temperature sensor 15, and the body temperature BTS detected by the second temperature sensing electrode (S81). Next, the operation unit 13 respectively determines whether the contact signal CS is abnormal, whether the ambient temperature TS is abnormal, and whether the body temperature BTS is abnormal (S82). If the contact signal CS, the ambient temperature TS and/or the body temperature BTS is abnormal, the operation unit 13 sends a reminding signal through the reminder unit 14 (S83).

If the contact signal CS, the ambient temperature TS and the body temperature BTS are within preset ranges, the operation unit 13 works out a first compensation parameter C1 according to the contact signal CS (S84). Next, the operation unit 13 works out first compensation bioinformatics CBS1 according to the first compensation parameter C1 (S85). The calculation methods of the step S84 and the step S85 are the same as those of the step S54 and the step S55 and will not repeat herein.

Next, the operation unit 13 works out a second compensation parameter C2 and a third compensation parameter C3 respectively according to the ambient temperature TS and the body temperature BTS (S86). The calculation methods of the second compensation parameter C2 and the third compensation parameter C3 are the same as those of the step S66 and the step S76 and will not repeat herein.

Next, the operation unit 13 works out a fourth compensation bioinformatics CBS4 according to the first compensation bioinformatics CBS 1, the second compensation parameter C2 and the third compensation parameter C3 (S87). The fourth compensation bioinformatics CBS4 may be expressed by Equation (7):

CBS4=CBS1×C3−C2 (7)

In conclusion, the present invention provides a bioinformatics sensor patch, which uses a distance sensing element to sense a contact degree between the bioinformatics sensor patch and an organism, whereby to compensate the sensed bioinformatics or remind the user to eliminate the abnormality, such as attaching the bioinformatics sensor patch once again, wherefore the measurement accuracy of the bioinformatics sensor patch is increased. Besides, the bioinformatics sensor patch of the present invention uses a first temperature sensor and/or a second temperature sensing electrode to sense the ambient temperature and/or the body temperature of the organism, whereby to compensate the sensed bioinformatics or remind the user to eliminate the abnormality, wherefore the measurement accuracy of the bioinformatics sensor patch is further increased.

While the invention is susceptible to various modifications and alternative forms, a specific example thereof has been shown in the drawings and is herein described in detail. It should be understood, however, that the invention is not to be limited to the particular form disclosed, but to the contrary, the invention is to cover all modifications, equivalents, and alternatives falling within the appended claims.

What is claimed is:

1. A bioinformatics sensor patch, comprising:
a plurality of sensing electrodes, configured to contact an organism and sense bioinformatics of the organism;
a distance sensing element, configured to sense a contact degree between the bioinformatics sensor patch and the organism and generate a corresponding contact signal; and
an operation unit, electrically connected with the plurality of sensing electrodes and the distance sensing element to receive the bioinformatics and the contact signal, executing at least one of actions of compensating the bioinformatics and selectively outputting a control signal according to the contact signal.

2. The bioinformatics sensor patch according to claim 1, further comprising:
a reminder unit, electrically connected with the operation unit, and generating a reminding signal recognizable by a user according to the control signal output by the operation unit.

3. The bioinformatics sensor patch according to claim 2, wherein the reminder unit includes a display, a light, a buzzer, a vibration motor, or a communication interface.

4. The bioinformatics sensor patch according to claim 1, wherein the operation unit performs a bioinformatics compensation method, which includes:
working out a first compensation parameter according to the contact signal; and
processing the bioinformatics according to the first compensation parameter to acquire first compensation bioinformatics.

5. The bioinformatics sensor patch according to claim 1, further comprising:
a first temperature sensor, electrically connected with the operation unit, and configured to sense an ambient temperature of the bioinformatics sensor patch, wherein the operation unit performs compensation to the bioinformatics or selectively outputs the control signal according to the ambient temperature.

6. The bioinformatics sensor patch according to claim 5, wherein the operation unit performs a bioinformatics compensation method, which includes:
working out a first compensation parameter according to the contact signal;
processing the bioinformatics according to the first compensation parameter to acquire first compensation bioinformatics;
working out a second compensation parameter according to the ambient temperature; and
processing the first compensation bioinformatics according to the second compensation parameter to acquire second compensation bioinformatics.

7. The bioinformatics sensor patch according to claim 1, further comprising:
a second temperature sensing electrode, electrically connected with the operation unit, and configured to sense a body temperature of the organism, wherein the operation unit performs compensation to the bioinformatics or selectively outputs the control signal according to the body temperature.

8. The bioinformatics sensor patch according to claim 7, wherein the operation unit performs a bioinformatics compensation method, which includes:
working out a first compensation parameter according to the contact signal;
processing the bioinformatics according to the first compensation parameter to acquire first compensation bioinformatics;
working out a third compensation parameter according to the body temperature of the organism; and
processing the first compensation bioinformatics according to the third compensation parameter to acquire third compensation bioinformatics.

9. The bioinformatics sensor patch according to claim 1, further comprising:
a first temperature sensor, electrically connected with the operation unit, and configured to sense an ambient temperature of the bioinformatics sensor patch; and
a second temperature sensing electrode, electrically connected with the operation unit, and configured to sense a body temperature of the organism, wherein the operation unit performs compensation to the bioinformatics or selectively outputs the control signal according to the ambient temperature and the body temperature.

10. The bioinformatics sensor patch according to claim 9, wherein the operation unit performs a bioinformatics compensation method, which includes:
working out a first compensation parameter according to the contact signal;
processing the bioinformatics according to the first compensation parameter to acquire first compensation bioinformatics;
working out a second compensation parameter and a third compensation parameter respectively according to the ambient temperature and the body temperature; and
processing the first compensation bioinformatics according to the second compensation parameter and the third compensation parameter to acquire fourth compensation bioinformatics.

11. The bioinformatics sensor patch according to claim 1, wherein the distance sensing element senses the contact degree between the bioinformatics sensor patch and the organism according to variation of resistance, capacitance, or voltage.

12. The bioinformatics sensor patch according to claim 1, wherein the sensing electrode is made of an electric-conduction material including at least one of graphene, carbon, gold, silver, stainless steel, nickel, nickel alloys, titanium, and titanium alloys.

13. The bioinformatics sensor patch according to claim 1, wherein a sensing terminal of the sensing electrode is in form of a sharp needle, a convex point or a plane.

14. The bioinformatics sensor patch according to claim 1, wherein the plurality of sensing electrodes includes at least one reference electrode, at least one working electrode and at least one counter electrode.

15. The bioinformatics sensor patch according to claim 1, wherein the plurality of sensing electrodes includes at least one reference electrode and a plurality of working electrodes.

16. The bioinformatics sensor patch according to claim 1, wherein the plurality of sensing electrodes includes at least one reference electrode and a plurality of working electrodes, and surfaces of the plurality of working electrodes are coated with different sensing layers.

17. The bioinformatics sensor patch according to claim 1, wherein the bioinformatics includes at least one of concentrations of blood glucose, blood ketone, lactic acid, an antibody, and a drug.
